(19) United States
(12) Patent Application Publication
(10) Pub. No.: US 2025/0214010 A1
(43) Pub. Date: Jul. 3, 2025

(54) CERAMIC CATALYTIC FILTER, METHOD OF MANUFACTURING THE SAME, PLASMA-CATALYST COMPOSITE SYSTEM, AND AIR PURIFICATION SYSTEM INCLUDING THE PLASMA-CATALYST COMPOSITE SYSTEM

(71) Applicant: Samsung Electronics Co., Ltd., Suwon-si (KR)

(72) Inventors: Su Keun KUK, Suwon-si (KR); Jaiyoung CHUNG, Suwon-si (KR); Hyun Chul LEE, Suwon-si (KR); Joonseon JEONG, Suwon-si (KR)

(21) Appl. No.: 19/004,376

(22) Filed: Dec. 29, 2024

(30) Foreign Application Priority Data
Dec. 29, 2023 (KR) 10-2023-0197699

(51) Int. Cl. B01D 39/20; A61L 9/16; B01J 23/889; B01J 35/30; B01J 35/45; B01J 35/57; B01J 35/63; B01J 35/64

(52) U.S. Cl. CPC B01D 39/2068; A61L 9/16; B01J 23/8892; B01J 35/395; B01J 35/45; B01J 35/57; B01J 35/638; B01J 35/651; B01D 2255/2073; B01D 2255/20761; B01D 2255/9022; B01D 2255/9155; B01D 2255/9202; B01D 2255/9205; B01D 2255/9207; B01D 2257/708; B01D 2259/818

(57) ABSTRACT

A ceramic catalytic filter includes a ceramic support, and a catalyst coating layer directly disposed on the ceramic support through a one-pot reaction. The catalyst coating layer includes a nanostructure.

This application claims priority to Korean Patent Application No. 10-2023-0197699, filed on Dec. 29, 2023, and all the benefits accruing therefrom under 35 U.S.C. § 119, the content of which in its entirety is herein incorporated by reference.

### BACKGROUND

### 1. Field

The disclosure relates to a ceramic catalytic filter, a method of manufacturing the same, a plasma-catalyst composite system including the same, and an air purification system.

### 2. Description of the Related Art

Volatile organic compounds (“VOCs”) are precursor materials of fine dust, and VOC reduction is desired to reduce fine dust. In the case of VOC reduction, adsorption-based removal methods using activated carbon or the like are used, but replacement after a certain amount of filtering is desired. Therefore, for sustainable VOC removal, many VOC decomposition technologies using thermal catalysts and photocatalysts are being researched. Such VOC removal catalysts are used to remove NOx by being applied onto ceramic filters through dip coating using a sol-gel method or a method using a binder for attaching a catalyst after the catalyst is synthesized.

### SUMMARY

A feature provides a ceramic catalytic filter, a method of manufacturing the same, a plasma-catalyst composite system including the same, and an air purification system.

Additional features will be set forth in part in the description which follows and, in part, will be apparent from the description, or may be learned by practice of the presented embodiments of the disclosure.

In an embodiment of the disclosure, there is provided a ceramic catalytic filter including a ceramic support, and a catalyst coating layer directly grown on the ceramic support through a one-pot reaction. The catalyst coating layer includes a nanostructure.

In an embodiment of the disclosure, there is provided a method of manufacturing a ceramic catalytic filter, the method including directly growing a catalyst coating layer on a ceramic support through a one-pot reaction.

In an embodiment of the disclosure, there is provided a plasma-catalyst composite system including the ceramic catalyst filter.

In an embodiment of the disclosure, there is provided an air purification system including the plasma-catalyst composite system, and an energy source disposed to supply energy for catalyst activation to the ceramic catalytic filter.

### BRIEF DESCRIPTION OF THE DRAWINGS

The above and other features and advantages of illustrative embodiments will be more apparent from the following description taken in conjunction with the accompanying drawings, in which:

FIG. 1 is a schematic three-dimensional view illustrating an embodiment of a shape of a ceramic catalytic filter;

FIG. 2 is a schematic cross-sectional view illustrating an embodiment of a plasma-catalyst composite system;

FIGS. 3 and 4 each show an embodiment of scanning electron microscope (“SEM”) images showing a morphology of a catalyst coating layer of a ceramic catalytic filter;

FIGS. 5 and 6 each show results of an embodiment of energy dispersive spectroscopy (“EDS”) elemental mapping of a ceramic catalytic filter;

FIG. 7 shows results of an embodiment of toluene removal of a plasma-catalyst composite system to which a ceramic catalytic filter is applied; and

FIG. 8 shows an embodiment of the distribution of toluene pores of a plasma-catalyst composite system to which a ceramic catalytic filter is applied.

### DETAILED DESCRIPTION

Reference will now be made in detail to embodiments, illustrative embodiments of which are illustrated in the accompanying drawings, wherein like reference numerals refer to like elements throughout. In this regard, the illustrated embodiments may have different forms and should not be construed as being limited to the descriptions set forth herein. Accordingly, the embodiments are merely described below, by referring to the drawing figures, to explain features of the description. As used herein, the term “and/or” includes any and all combinations of one or more of the associated listed items. Expressions such as “at least one of,” when preceding a list of elements, modify the entire list of elements and do not modify the individual elements of the list.

Hereinafter, as the inventive concept allows for various changes and numerous embodiments, illustrative embodiments will be illustrated in the drawings and described in detail in the written description. However, this is not intended to limit the inventive concept to particular modes of practice, and it is to be appreciated that all changes, equivalents, and substitutes that do not depart from the spirit and technical scope are encompassed in the inventive concept.

The terms used herein are merely used to describe illustrative embodiments and are not intended to limit the inventive concept. An expression used in the singular may encompass the expression of the plural, unless it has a clearly different meaning in the context. As used herein, it is to be understood that the terms such as “including,” “having,” and “comprising” are intended to indicate the existence of the features, numbers, steps, actions, components, parts, ingredients, materials, or combinations thereof disclosed in the specification and are not intended to preclude the possibility that one or more other features, numbers, steps, actions, components, parts, ingredients, materials, or combinations thereof may exist or may be added. The symbol “/” used herein may be interpreted as “and” or “or” according to the context.

In the drawings, the thicknesses of layers and regions are exaggerated or reduced for clarity. Like reference numerals in the drawings denote like elements throughout the specification. Throughout the specification, it will be understood that when a component, such as a layer, a film, a region, or a plate, is referred to as being “on” another component, the component may be directly on the other component or intervening components may be present thereon. Throughout the specification, while such terms as “first,” “second,” and “third,” and the like may be used to describe various components, such components should not be limited to the above terms. The above terms are used only to distinguish one component from another.

The term used herein is intended to describe only an illustrative embodiment and is not intended to limit the inventive concept. The term “or” refers to “and/or.” As used herein, the term “and/or” includes any and all combinations of one or more of associated listed items. It will be further understood that the terms “comprise” and/or “comprising” or “include” and/or “including” when used in this specification, specify the presence of stated features, regions, integers, steps, operations, and elements.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). The term “about” can mean within one or more standard deviations, or within ±30%, 20%, 10%, 5% of the stated value, for example.

Unless otherwise defined, all terms (including technical and scientific terms) used herein have the same meaning as commonly understood by one or ordinary skill in the art to which the disclosure belongs. In addition, it will be further understood that terms, such as those defined in commonly used dictionaries, should be interpreted as having a meaning that is consistent with their meaning in the context of the relevant art and the disclosure and will not be interpreted in an idealized sense unless expressly so defined herein. Also, the terms should not be interpreted in an overly formal sense.

Embodiments are described herein with reference to cross-sectional views which are schematic views of idealized embodiments. As such, variations from the shapes of the illustrations as a result manufacturing techniques and/or tolerances, are to be expected. Thus, the embodiments described in the disclosure should not be construed as limited to the particular shapes of regions of illustrated herein but may include deviations in shapes that result, for example, from manufacturing. For example, regions illustrated or described as being flat may be typically rough and/or have nonlinear features. Moreover, sharp angles that are illustrated may be rounded. Thus, regions illustrated in the drawings are schematic in nature and their shapes are not intended to illustrate the actual shape of a region and are not intended to limit the scope of the claims.

Hereinafter, a ceramic catalytic filter, a method of manufacturing the same, and an air purification system including the ceramic catalytic filter in embodiments will be described in more detail.

### [Ceramic Catalytic Filter]

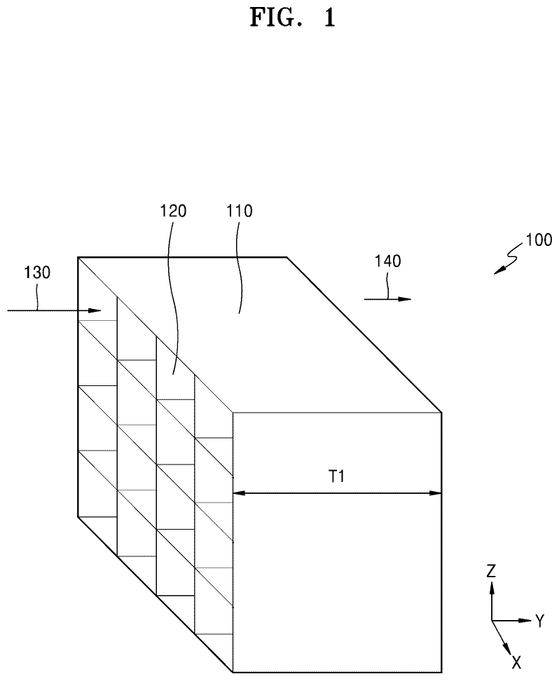

Referring to FIG. 1, a ceramic catalytic filter 100 in an embodiment may include a ceramic support 110, and a catalyst coating layer directly grown on the ceramic support 110 through a one-pot reaction. As used herein, the term “one-pot reaction” refers to a reaction whereby a reactant is subjected to successive chemical reactions in a single reactor.

In FIG. 1, although the ceramic support 110 is illustrated as defining a quadrangular groove, e.g., a rectangular groove, the shape of the ceramic support 110 is not limited thereto, and the ceramic support 110 may have various shapes. In an embodiment, a cross section of the ceramic support 110 may have various shapes such as a circular shape, an oval shape, a quadrangular shape such as a rectangular shape or a square shape, or other polygonal shapes, for example. In addition, the shape of the ceramic support 110 may be a cylindrical shape, a quadrangular parallelepiped shape, e.g., a rectangular parallelepiped shape, or a cubic shape with a height and diameter ranging from several millimeters (mm) to hundreds of millimeters (mm), but is not limited thereto.

In an embodiment, the ceramic support 110 may have a porous honeycomb structure.

In an embodiment, the ceramic support 110 may include or consist of magnesium oxide, silicon oxide, and aluminum oxide in a content of about 50% or more. In an embodiment, the ceramic support 110 may further include an alkaline oxide component. In embodiments, the alkaline oxide component may include Li₂O, Na₂O, K₂O, or the like. The ceramic support 110 further including or consisting of the alkali oxide component may maintain a shape of a ceramic catalytic filter without thermal deformation even at a substantially high temperature. The ceramic support 110 may be provided as a single layer or a plurality of stacks or may be provided as a single structure.

The ceramic catalytic filter 100 may be formed through a one-pot reaction and may be manufactured by applying a catalyst coating layer with a uniform nanostructure to have a substantially large area through a simple process. In addition, the catalyst coating layer may be formed uniformly. In addition, the nanostructure of the catalyst coating layer may include nanopores.

In an embodiment, when the catalyst coating layer is synthesized on the ceramic support 110, the catalyst coating layer may be simultaneously applied onto the ceramic support 110. A method of manufacturing the ceramic catalytic filter 100 may be described with reference to those described herein.

Referring to FIG. 1, the ceramic catalytic filter 100 may include an inlet side through which an air flow 130 flows in and an outlet side through which a gas 140 flows out. In an embodiment, the air flow 130 may include a particulate first material and a gaseous second material. The ceramic catalytic filter 100 may have a thickness T1 in a direction (Y-axis direction) in which the gas 140, which is generated as a result of a catalytic reaction between the ceramic catalytic filter 100 and a portion of the air flow 130 including a volatile organic compound (VOC), is discharged. A plurality of first grooves 120 may be defined in the ceramic catalytic filter 100, and the plurality of first grooves 120 may have an inlet in a direction in which the air flow 130 flows and a bottom in a direction (Y-axis direction) opposite to the direction. The air flow 130 flows into the ceramic catalytic filter 100 through the plurality of first grooves 120. The plurality of first grooves 120 may be regularly arranged. The plurality of first grooves 120 may be arranged parallel to each other. The catalyst coating layer may be formed on at least one surface (e.g., at least one of upper, lower and side surfaces) of the plurality of first grooves 120.

In order to activate a catalyst included in the catalyst coating layer, a type of catalyst included in the catalyst coating layer may vary according to energy supplied to the ceramic catalytic filter 100.

In an embodiment, when energy supplied to the ceramic catalytic filter 100 is light energy, the catalyst coating layer may include a metal compound capable of causing a photocatalytic reaction. In an embodiment, the metal compound may be at least one of TiO₂ or WO₃. The light energy may include ultraviolet light energy or visible light energy, for example.

In an embodiment, when energy supplied to the ceramic catalytic filter 100 is direct current (“DC”) or alternating current (“AC”) electrical energy, the catalyst coating layer may include a metal compound capable of causing an electrically conductive oxygen reduction reaction (“ORR”). In an embodiment, the metal compound may be a compound including a metal such as Co, Ni, or Mn, may include a noble metal oxide, or may include a combination of the metal and the noble metal oxide, for example.

In an embodiment, when energy supplied to the ceramic catalytic filter 100 is ion energy, the catalyst coating layer may include a metal compound capable of being oxidized by ozone. In an embodiment, the metal compound may be at least one of CuO, Cu₂O, MnO₂, CuMnOₓ, or ZnO₂. The ion energy may be, e.g., plasma energy, for example.

In an embodiment, when energy supplied to the ceramic catalytic filter 100 is thermal energy, the catalyst coating layer may include a metal compound capable of causing a low-temperature oxidation reaction. In an embodiment, the metal compound may be a compound including at least one of Cu, Co, Ni, Fe, Al, Si, or a noble metal. The low-temperature oxidation reaction may refer to an oxidation reaction that occurs in a temperature ranging from room temperature to about 100 degrees Celsius (° C.). The heat energy may include infrared energy, energy supplied from a heat source such as a heater, or any combinations thereof, for example.

In an embodiment, the catalyst coating layer may include a catalyst including or consisting of an oxide of a first metal, and the first metal may be manganese (Mn), copper (Cu), or any combinations thereof.

In an embodiment, the catalyst coating layer may include MnO₂ or CuMnOₓ, for example.

In an embodiment, an average thickness of the catalyst coating layer may be in a range of about 10 nm to about 1,000 nm. In an embodiment, the average thickness of the catalyst coating layer may be in a range of about 25 nm to about 750 nm, about 50 nm to about 500 nm, or about 100 nm to about 300 nm, for example.

A predetermined surface area of the catalyst coating layer may be in a range of about 20 m²/g to about 300 m²/g. In an embodiment, the predetermined surface area of the catalyst coating layer may be in a range of about 30 m²/g to about 250 m²/g or about 50 m²/g to about 230 m²/g, for example. By securing a substantially large surface area in such a range, a desired level of adsorption and decomposition efficiency of the VOC may be obtained. As the predetermined surface area of the catalyst coating layer is widened, the adsorption efficiency of the VOC may increase, and the decomposition efficiency thereof may also increase.

The ceramic catalytic filter 100 in an embodiment may include the catalyst coating layer, and the catalyst coating layer may include a nanostructure.

Nanopores may be defined in the catalyst coating layer by a nanostructure of a catalyst, and thus a reaction catalyst effect of the ceramic catalytic filter 100 may be improved. In an embodiment, when the ceramic catalytic filter 100 is applied to a plasma-catalyst composite system, an electric field may increase due to a nanostructure of a catalyst, and more micro-discharges may be generated in internal nanopores, for example, thereby improving a synergy effect between dielectric barrier discharge (“DBD”) plasma and the catalyst.

In an embodiment, the nanostructure may be a grain structure, a needle structure, a pillar structure, a wire structure, or any combinations thereof.

In an embodiment, the nanostructure may include two or more different structures.

In an embodiment, an average size of the nanostructure may be in a range of about 50 nm to about 1,000 nm. In an embodiment, the average size of the nanostructure may be in a range of about 100 nm to about 500 nm, about 150 nm to about 450 nm, or about 200 nm to about 400 nm, for example.

In an embodiment, pores may be defined in the catalyst coating layer, and a size of the pores may be in a range of about 0.01 nm to about 200 nm. In an embodiment, the size of the pores may be in a range of about 0.1 nm to about 100 nm or about 1 nm to about 50 nm. In such a range, an interval between catalyst materials may be narrowed, thereby improving a catalytic effect and further improving a synergy effect between plasma and a catalyst, for example.

In an embodiment, a volume of the pores may be in a range of about 10 cubic centimeters per gram (cm³/g) to about 50 cm³/g. In an embodiment, the volume of the pores may be in a range of about 12 cm³/g to about 30 cm³/g or about 15 cm³/g to about 20 cm³/g, for example. In such a range, the interval between the catalyst materials may be narrowed, thereby improving a catalytic effect and further improving a synergy effect between plasma and a catalyst.

The pores defined in the catalyst coating layer may promote the generation of micro-discharge when plasma energy is applied, and thus the efficiency of a plasma-catalyst composite system including the ceramic catalytic filter 100 may be further improved.

In an embodiment, the ceramic catalytic filter 100 may remove the VOC from the air flow 130 including the VOC.

In an embodiment, the VOC may be formaldehyde, acetaldehyde, ammonia, toluene, acetic acid, or any combinations thereof. In an embodiment, the VOC may be toluene, for example.

### [Method of Manufacturing Ceramic Catalytic Filter]

A method of manufacturing a ceramic catalytic filter in an embodiment may include directly growing a catalyst coating layer on a ceramic support through a one-pot reaction.

In an embodiment, in the direct growing of the catalyst coating layer, synthesizing of the catalyst coating layer and application of the catalyst coating layer onto the ceramic support may be simultaneously performed, for example.

In an embodiment, the application of the catalyst coating layer may not use a binder.

Through the method of manufacturing a ceramic catalytic filter, a ceramic catalytic filter in which the catalyst coating layer with a uniform nanostructure is uniformly applied to have a substantially large area may be manufactured through a simple process.

A catalyst coating method of a ceramic catalytic filter of a related art has several limitations. In an embodiment, in the case of a sol-gel dip coating, it is difficult to apply a catalyst thinly and it is difficult to control a shape and structure thereof, for example. In addition, due to the viscosity of a sol, the uniformity of the coating is low, which makes effective coating difficult. Such drawbacks make it difficult to fully implement the activity of a catalyst on a filter surface. In another embodiment, since coating using an organic binder is a method in which, after a catalyst is synthesized, the catalyst is attached to a filter surface by an adhesive organic binder, there is a limitation in that the number of process increases. In addition, there is a drawback in that the organic binder that is not sufficiently removed after a process may release harmful substances when thermally decomposed and/or photodecomposed.

In the method of manufacturing a ceramic catalytic filter in an embodiment, through a simple synthesis and coating process of a one-pot reaction method, without the use of a binder, a catalyst coating layer with a nanostructure may be uniformly applied onto outer and/or inner wall surfaces of a three-dimensional ceramic filter with a complex internal structure to have a substantially large area. In addition, since an additional binder or adhesive is not used, without the emission of harmful substances, a ceramic catalytic filter, which may be applied to cleaning, photolysis, and thermal decomposition, may be manufactured.

In an embodiment, the ceramic catalytic filter may be manufactured by preparing a coating solution including a catalyst precursor and then immersing the ceramic support in the coating solution to simultaneously perform the synthesis of a catalyst and the formation of a catalyst coating layer. In addition, the catalyst coating layer may be dried at a temperature ranging from about 70° C. to about 100° C., e.g., in a vacuum oven. In an embodiment, such heat treatment may be performed at a temperature ranging from about 400° C. to about 800° C., e.g., in a furnace, for example.

### [Plasma-Catalyst Composite System]

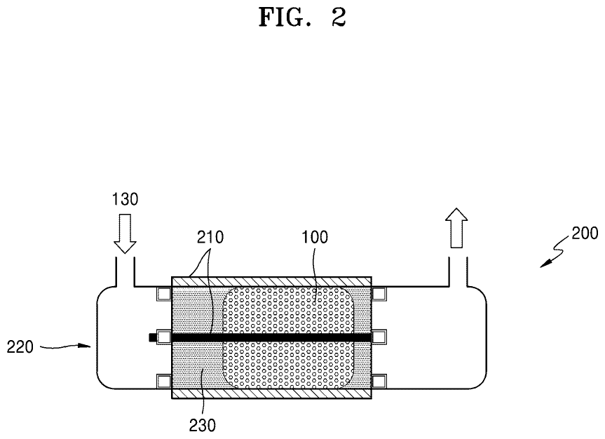

Referring to FIG. 2, a plasma-catalyst composite system 200 in an embodiment may include the ceramic catalytic filter 100.

In an embodiment, the plasma-catalyst composite system 200 may include a DBD plasma reactor 220.

The DBD may be a method in which, when a dielectric is inserted between a high-voltage electrode and a ground electrode and an alternating current (“AC”) voltage is applied to the high-voltage electrode, electrons are accelerated in an electric field region between the two electrodes to ionize injected gas and generate plasma. In order to generate plasma, there is no need to apply a substantially high voltage, and there is no need for a complicated pulse power supply, and plasma generation is easy through micro-discharge.

Referring to FIG. 2, the plasma-catalyst composite system 200 in an embodiment may include two electrodes 210, the DBD plasma reactor 220, and a plasma discharge region 230. In addition, as shown in FIG. 2, a gas supply unit and a gas outlet may be provided, and an air flow 130 including a VOC may be flow in through the gas supply unit, and the air flow 130 after a reaction may flow out through the gas outlet. In an embodiment, an inert gas may be added to facilitate the generation of plasma. The inert gas may include, e.g., helium, nitrogen, argon, or any combinations thereof.

In an embodiment, the ceramic catalytic filter 100 may be disposed in the plasma discharge region 230 in the DBD plasma reactor 220. In an embodiment, the plasma-catalyst composite system 200 may be an in-plasma catalyst (“IPC”) type plasma-catalyst composite system, for example.

In an embodiment, the ceramic catalytic filter 100 may be disposed outside the plasma discharge region 230 in the DBD plasma reactor 220. In an embodiment, the plasma-catalyst composite system 200 may be a post-plasma catalyst (“PPC”) type plasma-catalyst composite system, for example.

When a substantially high voltage may be applied to the electrodes 210 of the plasma-catalyst composite system 200 by a power supply, plasma may be generated from a reactive gas in the plasma discharge region 230 including a catalyst layer in the DBD plasma reactor 220, and a reaction in which the VOC is converted to CO and/or CO₂ may occur due to an interaction between plasma and a catalyst. The power supply may be an AC power source or a direct current (“DC”) power source.

### [Air Purification System]

An air purification system in an embodiment may include the plasma-catalyst composite system, and an energy source disposed to supply energy for catalyst activation to the ceramic catalytic filter.

In an embodiment, the energy source may include at least one of a light energy generator, an electrical energy generator, an ion energy generator, and a thermal energy generator, and the energy source may supply at least one of light energy, electrical energy, ion energy, and thermal energy.

Hereinafter, embodiments will be described in more detail with reference to the following Embodiments and Comparative Examples. However, Embodiments are not intended to limit the scope of the disclosure.

### EMBODIMENTS

### Manufacturing Embodiment 1

As a ceramic support, a cordierite ceramic material having a quadrangular shape with a length of 10 centimeter (cm) was prepared. A thickness of each cell of the ceramic support is in a range of 0.2 mm to 0.5 mm, and a distance between inner walls is in a range of 1 mm to 3 mm.

In order to synthesize a MnO₂ nanocatalyst direct-growth coating layer, in a reaction vessel, as catalyst precursors, 5.1 grams (g) of MnSO₄·H₂O, 6.5 g of KClO₃, and 1.7 milliliters (ml) of sulfuric acid were dissolved in 100 ml of a coating aqueous solution.

The ceramic support was immersed in a precursor aqueous solution contained inside a capped glass bottle and then allowed to react at a temperature 90 Celsius degrees (° C.) for 12 hours. During the reaction, stirring was performed by a stirring bar. After the reaction, a sufficiently cooled filter was taken out, put into water, and cleaned through sonication for 2 hours. Afterwards, the cleaned filter was dried at a temperature of 80° C. for 12 hours in a vacuum oven.

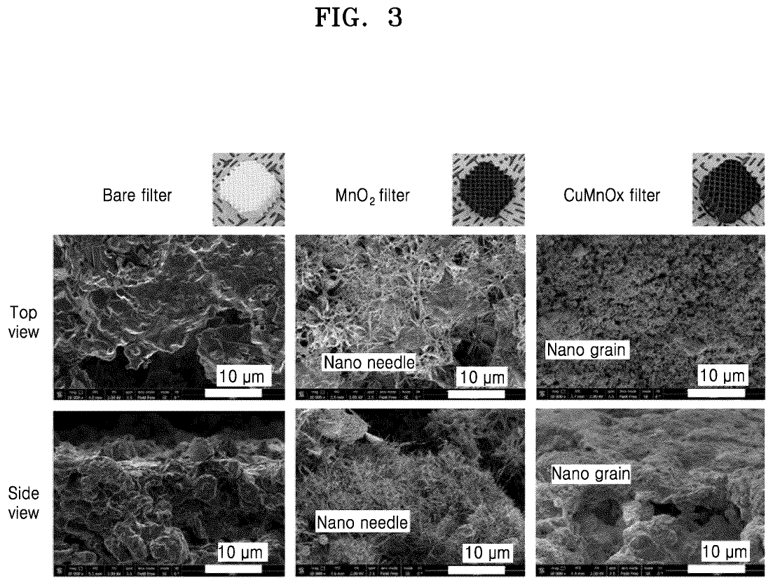

After heat treatment, a ceramic catalytic filter coated with a MnO₂ catalyst coating layer was obtained. Scanning electron microscope (“SEM”) images of the obtained ceramic catalytic filter are shown in FIG. 3. Referring to FIG. 3, it could be seen that the ceramic catalytic filter of Manufacturing Embodiment 1 had a needle-shaped nanostructure with an average length of 5.04 micrometers (μm).

### Manufacturing Embodiment 2

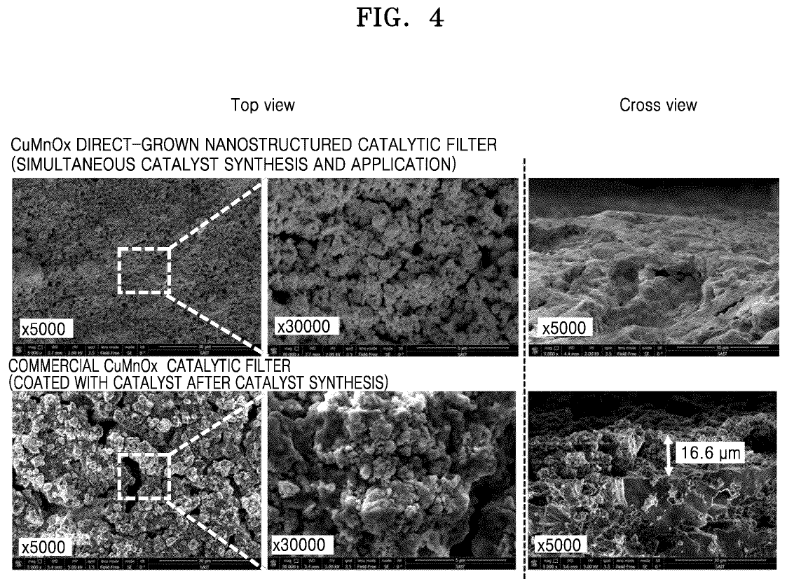

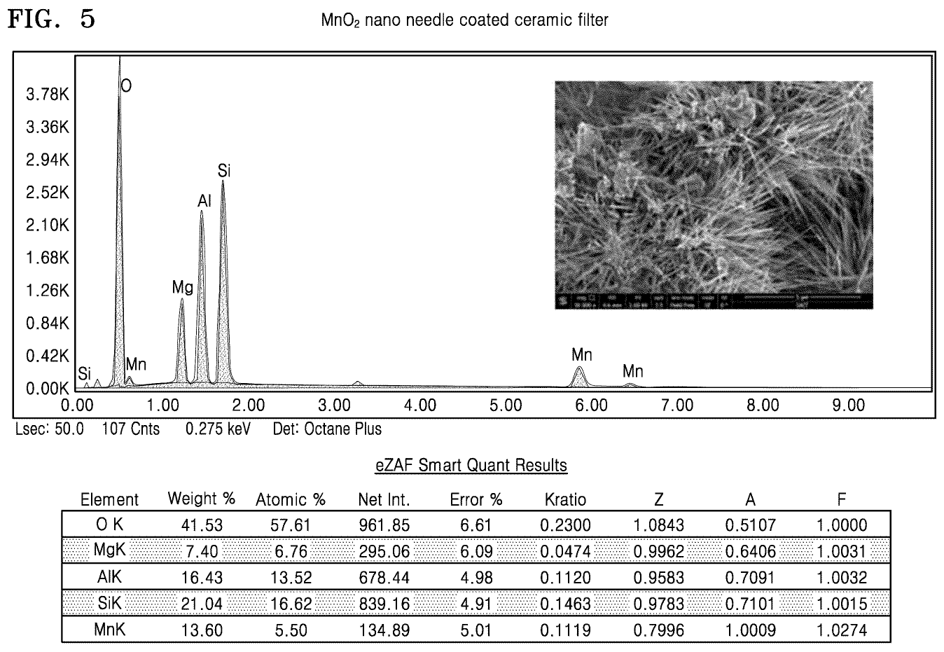

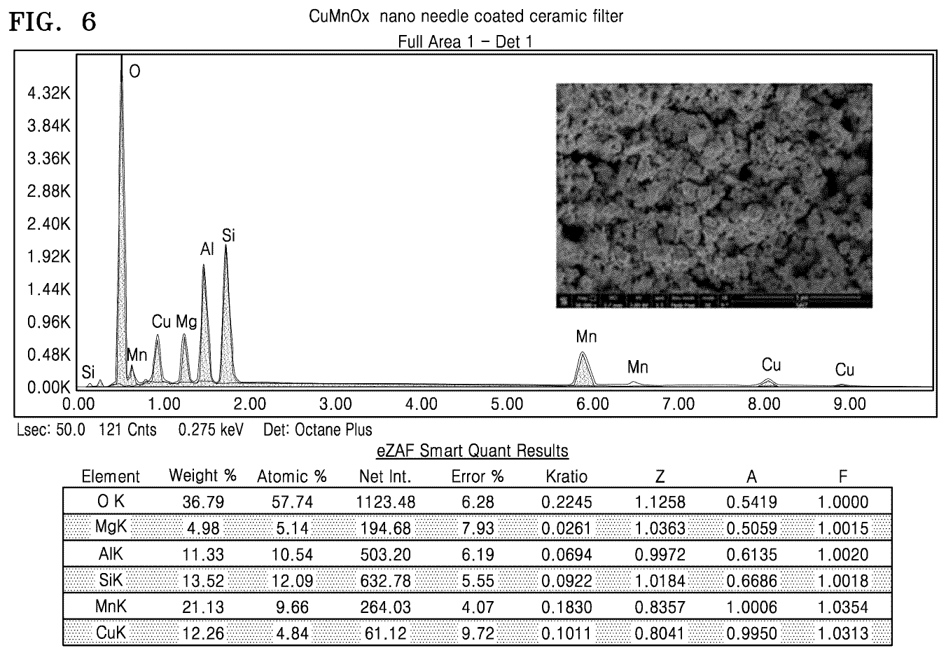

A ceramic catalytic filter coated with a CuMnOₓ catalyst coating layer was obtained in the same manner as in Manufacturing Embodiment 1, except that 6.3 g of KMnO₄ and 4.8 g of Cu(NO₃)₂·3H₂O were used as catalyst precursors instead of MnSO₄·H₂O, KClO₃, and sulfuric acid. SEM images of the obtained ceramic catalytic filter are shown in FIG. 4. Referring to FIG. 4, it could be seen that the ceramic catalytic filter of Manufacturing Embodiment 2 had a grain-shaped nanostructure with an average length of 380 nm. In addition, energy-dispersive X-ray spectroscopy (“EDS”) elemental mapping analysis was performed on the ceramic catalytic filters of Manufacturing Embodiment 1 and Manufacturing Embodiment 2. Results thereof are shown in FIGS. 5 and 6, respectively. Referring to FIGS. 5 and 6, it could be confirmed that the MnO₂ catalyst coating layer with a nanoneedle structure and the CuMnOₓ catalyst coating layer with a nanograin structure were well synthesized.

### <Evaluation 1: Toluene Decomposition Evaluation (1)>

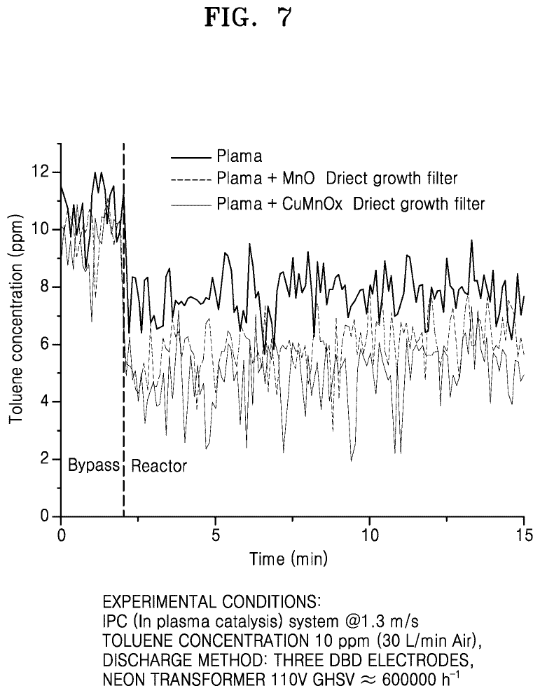

A toluene decomposition experiment was performed on plasma-catalyst composite systems to which the ceramic catalytic filters of Manufacturing Embodiment 1 and Manufacturing Embodiment 2, respectively. Results thereof are shown in FIG. 7 and Table 1. Specific conditions of evaluation are shown in FIG. 7.

TABLE 1

| | Toluene removal | | Amount of CO | | Amount of CO₂ | |
|---|---|---|---|---|---|---|
| | ratio | | production | | production | |
| | % | Ratio (%) | ppm | Ratio (%) | ppm | Ratio (%) |
| Plasma | 30.6 | 100 | 3.2 | 100 | 4.7 | 100 |
| Plasma + MnO₂ | 38.1 | 124.5 | 3.6 | 112.5 | 6.2 | 131.9 |
| DGF | | | | | | |
| Plasma + CuMnOₓ | 46.3 | 151.3 | 4.8 | 150 | 8.6 | 183.0 |
| DGF | | | | | | |

In Table 1, the term “DGF” denotes a direct growth filter.

Referring to FIG. 7 and Table 1, it was confirmed that, when a ceramic catalytic filter in an embodiment was applied to plasma, a larger amount of toluene was removed as compared with when only plasma was present. Thus, it could be seen that a coordination effect of the plasma and the catalytic filter was excellent in removing a VOC.

### <Evaluation 2: Evaluation of Pore Characteristics of Ceramic Catalytic Filter>

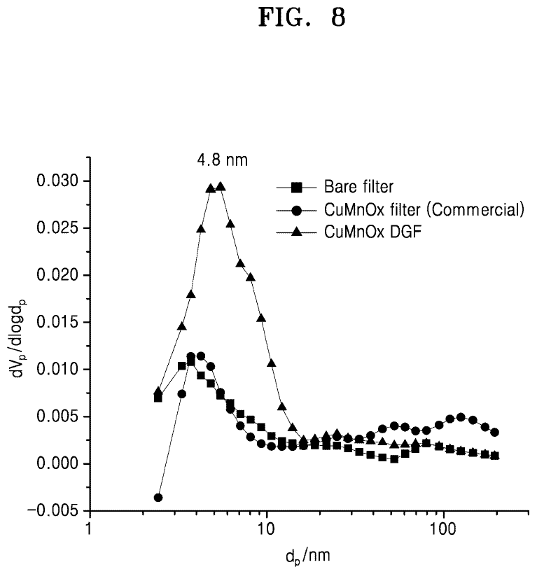

Through a Barrett-Joyner-Halenda (“BJH”) method using Brunauer-Emmett-Teller (“BET”) equipment, a distribution of pores was analyzed on each of the ceramic catalytic filter (CuMnOₓ DGF) of Manufacturing Embodiment 2, a commercial CuMnOₓ catalytic ceramic filter (commercial CuMnOₓ), and a ceramic support (bare filter) on which a catalyst coating layer was not formed. Results thereof are shown in FIG. 8. In addition, a volume and an area of the pores were analyzed. Results thereof are shown in Table 2.

TABLE 2

| | Pore volume (cm³g⁻¹) | Pore size (m²g⁻¹) |
|---|---|---|
| Bare filter | 7.5 × 10⁻³ | 6.4 |
| (Commercial) CuMnOₓ | 6.9 × 10⁻³ | 2.0 |
| filter | | |
| CuMnOₓ DGF | 16.5 × 10⁻³ | 12.3 |

Referring to FIG. 8 and Table 2, it could be seen that the number, volume, and area of the pores in the ceramic catalytic filter in an embodiment all increased.

### <Evaluation 3: Toluene Decomposition Evaluation (2)>

A toluene decomposition experiment was performed on each of the ceramic catalytic filter (CuMnOₓ DGF) of Manufacturing Embodiment 2, the commercial CuMnOₓ catalytic ceramic filter (commercial CuMnOₓ), and the ceramic support (bare filter) on which a catalyst coating layer was not formed. Results thereof are shown in Table 3. predetermined evaluation conditions are the same as in Evaluation 1.

TABLE 3

| | Toluene removal | | Amount of CO | | Amount of CO₂ | |
|---|---|---|---|---|---|---|
| | ratio | | production | | production | |
| | | Ratio | | Ratio | | Ratio |
| | % | (%) | ppm | (%) | ppm | (%) |
| Plasma | 30.6 | 100 | 3.2 | 100 | 4.7 | 100 |
| Plasma + (commercial) | 38.6 | 126.1 | 3.6 | 112.5 | 6.3 | 134.0 |
| CuMnOₓ | | | | | | |
| Plasma + CuMnOₓ | 46.3 | 151.3 | 4.8 | 150 | 8.6 | 183.0 |
| DGF | | | | | | |

Referring to Table 3, it could be seen that, in the ceramic catalytic filter in an embodiment, a toluene removal ratio was higher and amounts of CO production and CO₂ production were larger as compared with the commercial ceramic filter. That is, it could be seen that, in the ceramic catalytic filter in an embodiment, the number of manufacturing processes decreased by applying a one-pot reaction method to a porous ceramic support, and at the same time, a VOC decomposition ability was higher as compared with the commercial ceramic filter. In addition, in combination with the results of Evaluation 2, it could be seen that, since the catalytic filter layer of the ceramic catalytic filter in an embodiment included a nanostructure, an electric field increased, and the generation of micro-discharge increased due to internal pores of the filter layer, thereby further improving a plasma-catalyst synergy effect.

While embodiments have been described in detail with reference to the accompanying drawings, the inventive concept is not limited to the embodiments. It is obvious to those skilled in the art to which the inventive concept belongs that various changes and modifications are conceivable within the scope of the technical idea described in the claims, and those are understood as naturally belonging to the technical scope of the inventive concept.

The ceramic catalytic filter may be manufactured by applying a catalyst coating layer with a uniform nanostructure to have a substantially large area through a simple process of a one-pot reaction. In addition, the catalyst coating layer may be formed uniformly.

In addition, nanopores may be defined in the catalyst coating layer by a nanostructure of a catalyst, and thus a reaction catalyst effect of the ceramic catalytic filter may be improved. In an embodiment, when the ceramic catalytic filter is applied to a plasma-catalyst composite system, an electric field may increase due to a nanostructure of a catalyst, and more micro-discharges may be generated in internal nanopores, thereby improving a synergy effect between DBD plasma and the catalyst, for example.

Therefore, a highly efficient plasma-catalyst composite system and air purification system may be implemented by the ceramic catalytic filter.

It should be understood that embodiments described herein should be considered in a descriptive sense only and not for purposes of limitation. Descriptions of features or advantages within each embodiment should typically be considered as available for other similar features or advantages in other embodiments. While embodiments have been described with reference to the drawing figures, it will be understood by those of ordinary skill in the art that various changes in form and details may be made therein without departing from the spirit and scope as defined by the following claims.

What is claimed is:

1. A ceramic catalytic filter comprising:
a ceramic support; and
a catalyst coating layer directly grown on the ceramic support through a one-pot reaction, the catalyst coating layer comprising a nanostructure.

2. The ceramic catalytic filter of claim 1, wherein the catalyst coating layer comprises a catalyst including an oxide of a first metal,
wherein the first metal includes manganese (Mn), copper (Cu), or a combination thereof.

3. The ceramic catalytic filter of claim 1, wherein the catalyst coating layer has a thickness of about 10 nanometers to about 1,000 nanometers.

4. The ceramic catalytic filter of claim 1, wherein, in response to synthesizing of the catalyst coating layer on the ceramic support, the catalyst coating layer is simultaneously applied onto the ceramic support.

5. The ceramic catalytic filter of claim 1, wherein the ceramic support has a porous honeycomb structure.

6. The ceramic catalytic filter of claim 1, wherein the nanostructure is a grain structure, a needle structure, a pillar structure, a wire structure, or any combinations thereof.

7. The ceramic catalytic filter of claim 6, wherein the nanostructure comprises two or more different structures.

8. The ceramic catalytic filter of claim 1, wherein the nanostructure has an average size of about 50 nanometers to about 1,000 nanometers.

9. The ceramic catalytic filter of claim 1, wherein the catalyst coating layer comprises pores,
wherein the pores have a size of about 0.01 nanometers to about 200 nanometers.

10. The ceramic catalytic filter of claim 9, wherein the pores have a volume of about 10 cubic centimeters per gram to about 50 cubic centimeters per gram.

11. The ceramic catalytic filter of claim 1, wherein the ceramic catalytic filter removes volatile organic compounds from an unpurified air flow including the VOCs.

12. The ceramic catalytic filter of claim 11, wherein the volatile organic compounds comprise toluene.

13. A method of manufacturing a ceramic catalytic filter, the method comprising:
directly growing a catalyst coating layer on a ceramic support through a one-pot reaction.

14. The method of claim 13, wherein, in the direct growing the catalyst coating layer, synthesizing of the catalyst coating layer and application of the catalyst coating layer onto the ceramic support are performed simultaneously.

15. The method of claim 14, wherein the application of the catalyst coating layer does not use a binder.

16. A plasma-catalyst composite system comprising the ceramic catalytic filter of claim 1.

17. The plasma-catalyst composite system of claim 16, further comprising a dielectric barrier discharge plasma reactor,
wherein the ceramic catalytic filter is disposed in a plasma discharge region in the dielectric barrier discharge plasma reactor.

18. The plasma-catalyst composite system of claim 17, wherein the ceramic catalytic filter is disposed in the plasma discharge region in the dielectric barrier discharge plasma reactor.

19. An air purification system comprising the plasma-catalyst composite system of claim 16.

20. The air purification system of claim 19, further comprising an energy source which supplies energy for catalyst activation to the ceramic catalytic filter,
wherein the energy source supplies at least one of light energy, electrical energy, ion energy, and thermal energy.
